(19) United States
(12) Patent Application Publication
(10) Pub. No.: US 2009/0112080 A1
(43) Pub. Date: Apr. 30, 2009

(54) System for Measuring Electric Signals

(75) Inventor: Robert Matthews, San Diego, CA (US)

Correspondence Address: DIEDERIKS & WHITELAW, PLC, 12471 DILLINGHAM SQUARE, #301, WOODBRIDGE, VA, 22192, US

(73) Assignee: QUANTUM APPLIED SCIENCE & RESEARCH, INC., San Diego, CA (US)

(21) Appl. No.: 12/298,002

(22) PCT Filed: Apr. 23, 2007
(86) PCT No.: PCT/US2007/009748

§ 371 Date: Oct. 21, 2008

Related U.S. Application Data
(60) Provisional application No. 60/794,275, filed on Apr. 21, 2006.

(51) Int. Cl. A61B 5/04

(52) U.S. Cl. 600/393

(57) ABSTRACT

A system for determining if signals present at bioelectric sensors derive from an intended source or from different, localized sources or artifacts includes a first sensor placed to detect the electric potential of interest and generate a first electric signal possibly representative of the electric potential of interest and a second sensor placed near the first sensor and preferably a relatively large distance away from the source. The second sensor detects the electrical potential of interest and generates a second electrical signal which also possibly represents the electrical potential of interest. An electronic circuit determines whether a difference between the electrical signals exceeds a certain threshold, thus indicating that either one or both of the signals is a measure of an artifact and not the electric potential of interest.

### CROSS-REFERENCE TO RELATED APPLICATIONS

The present application represents a National Stage application of pending PCT/US2007/009748 filed Apr. 23, 2007 entitled “System for Measuring Electric Signals”, and further claims the benefit of U.S. Provisional Patent Application Ser. No. 60/794,275 filed Apr. 21, 2006 entitled “ECG Monitoring System”.

### STATEMENT REGARDING FEDERALLY SPONSORED RESEARCH AND DEVELOPMENT

The U.S. Government has a paid-up license in this invention and the right in limited circumstances to require the patent owner to license others on reasonable terms as provided for by the terms of Phase II SBIR Contract No. W31P4Q-04-C-R293 awarded by DARPA.

### BACKGROUND OF THE INVENTION

1. Field of the Invention

The present invention pertains to the art of measuring electric signals and, more particularly, to a system for measuring bioelectric signals that determines whether detected signals derive from a source of interest or result from an artifact or other undesired interference in acquired data.

2. Discussion of the Prior Art

It is widely known that electric fields are developed in free space from many different sources. For example, organs in the human body, including the heart and brain, produce electric fields. For a variety of reasons, it is often desirable to measure these electric fields, such as in performing an electrocardiogram (ECG). Actually, the measurement of bioelectric signals can provide critical information about the physiological status and health of an individual, and is widely used in monitoring, evaluating, diagnosing and caring for patients, as well as providing feedback for athletic training. Common methods of measuring electric potentials associated with a human employ gel-coated electrodes that must be secured directly to the skin of a subject. In addition, in recent years a number of alternate electrode technologies have been developed. While the alternate electrode techniques enable more convenient and comfortable measurement configurations, they are often prone to measurement artifacts.

More specifically, resistive electrodes have been predominantly employed in connection with measuring electric potentials produced by animals and human beings. As the resistive electrodes must directly touch the skin, preparation of the skin to achieve an adequate resistive connection is required. Such resistive electrodes are the standard for current medical diagnostics and monitoring, but the need for skin preparation and contact rule out expanding their uses. Although attempts have been made to construct new types of resistive electrodes, such as making an electrically conductive fabric, providing a miniature grid of micro-needles that penetrate the skin, and developing chest belt configurations for heart related measurements or elasticized nets with resistive sensors making contact via a conductive fluid for head-related measurements, these alternative forms do not overcome the fundamental limitation of needing to contact the skin directly. This limitation leads to an additional concern regarding the inability to maintain the necessary electrical contact based on differing physical attributes of the patient, e.g. amount of surface hair, skin properties, etc.

Another type of sensor that can be used in measuring biopotentials is a capacitive sensor. Early capacitive sensors required a high mutual capacitance to the body, thereby requiring the sensor to touch the skin of the patient. The electrodes associated with these types of sensors were strongly affected by lift-off from the skin, particularly since the capacitive sensors were not used with conducting gels. As a result, capacitive sensors were not found to provide any meaningful benefits and were not generally adopted over resistive sensors. However, advances in electronic amplifiers and new circuit techniques have made possible a new class of capacitive sensor that can measure electrical potentials when coupling to a source on the order of 1 pF or less. Examples of low noise electric field sensors can be found in U.S. Pat. Nos. 6,686,800 and 7,088,175, each of which is incorporated herein by reference. This capability makes possible the measurement of bioelectric signals with electrodes that do not need a high capacitance to the subject, thereby enabling the electrodes to be used without being in intimate contact with the subject.

Substantial body motion during exercise or daily life can produce artifacts in any bioelectric measurement system, but these effects become more pronounced with many alternate electrode technologies. These artifacts are caused by mechanisms that are local to the skin and sensor, such as static electric potentials, electromyographic signals and piezoelectric artifacts. In contrast, the signal produced by the heart, brain or other organ originates within the body at a much greater distance from the sensor. Hence, there exists a need to determine when the data reflects a distant source, or results from local sources. Some such methods attempt to confirm that the data have the general structure expected. However, these may reject valid signals having non-standard features, and may accept artifact data that happen to conform to the expected model. Therefore, it is desirable to determine if data are valid independently of the specific data themselves.

Therefore, there exists a need in the art for a system that can determine when the data taken by a bioelectric measurement system reflect a distant source, or result from local artifacts. There also exists a need for less intrusive electrode technology, together with the capability to tolerate artifacts at the input, and allow for measurement configurations which were previously not practical.

### SUMMARY OF THE INVENTION

The present invention is directed to a system for discerning the validity of bioelectric data and adds to a basic measurement system one or more additional sensors that measure related signals. Based on known relations between the sensors and a primary source of the bioelectric signals, and on known response characteristics of the sensors, a relation of the expected signals produced by the source on the multiple sensors can be predicted. If the observed signals do not show the expected relation, then it is concluded that a significant part of the measured signal from at least one of the sensors compared must derive from a cause other than the primary source, and therefore is due to sources local to the sensor. For example, if two sensors are very close to each other and comparatively far from the signal source, the signals they measure from that source should be similar. Any difference in the two signals must therefore come from some local source. If the difference is significant, then the data measured during the period in which the difference appears are suspect.

The invention generally includes a sensor system for measuring an electric potential of interest generated by a source such as the heart or brain of the human body. A first sensor is placed at a first measurement location to detect the electric potential of interest and generate a first electric signal possibly representative of the electric potential of interest. A second sensor is placed at a second measurement location near the first sensor and preferably a relatively large distance away from the source. The second sensor detects the electrical potential of interest and generates a second electrical signal which also possibly represents the electrical potential of interest. Optionally, an adjuster or circuitry is provided for altering the first and second electrical signals to compensate for changes caused by placement of the sensors or the electrical characteristics of the sensors themselves. For example, the adjuster could adjust the gain or amplification of the signal produced by each sensor. In another embodiment, the adjuster involves filtering the data from one sensor to account for known changes in the source signal between first and second measurement locations should a sensor be moved from the second measurement location to the first measurement location.

A comparator compares the first and second signals to produce a comparison result representing a measurement of that difference. An electronic circuit determines whether the comparison result exceeds a certain threshold level, thus indicating that either one or both of the signals is a measure of an artifact and not the electric potential of interest.

The threshold level may either be static or dynamic. In one embodiment, the comparator compares the magnitude of the signals. In another embodiment, it checks for a time offset of the signals produced by each of the two sensors.

The sensor system may incorporate various different types of devices. In one preferred embodiment, the sensor system is incorporated into an audio generating device. In another embodiment, the sensor system is enclosed and the sensors themselves are in the arms or shoulders of a garment. In yet another embodiment, the sensor system is independent of the garment and attached thereto through some type of connection mechanism and, as such, is removably attached to the garment.

In use, the sensor system measures electrical signals by using the sensors, then adjusts the signals to compensate for placement and/or electrical characteristics of the signals. The two signals are then compared to see whether or not the differences in the signals reach a threshold to determine whether or not the signals are caused by a local artifact or the distant source.

In yet another embodiment, one or more additional sensors are used in the system. For example, a third sensor may be used and signals generated by the third sensor analyzed and compared with each of the first two sensors as described above in regards to the first and second sensors. The resulting signal from each additional pair of sensors considered valid may be averaged in with the signal from the existing sensors to obtain a more accurate final signal.

Additional objects, features and advantages of the present invention will become more readily apparent from the following detailed description of preferred embodiments when taken in conjunction with the drawings wherein like reference numerals refer to corresponding parts in the several views.

### BRIEF DESCRIPTION OF THE DRAWINGS

FIG. 1 schematically illustrates a system for measuring electric signals according to a preferred embodiment of the invention;

FIG. 2 is a graph depicting two electrocardiograms measured with closely spaced sensors;

FIG. 3 is a graph depicting the electrocardiogram of FIG. 2 and also showing the magnitude of the difference between the signals;

FIG. 4 is a graph depicting the electrocardiogram of FIG. 2 and also showing regions of traces identified as an artifact;

FIG. 5 is a graph depicting the electrocardiogram of FIG. 4 and also showing regions of traces identified as an artifact having been removed;

FIG. 6 schematically illustrates the system for measuring electrical systems incorporated with the sleeves of a garment;

FIG. 7 schematically illustrates the system for measuring electrical systems incorporated with the shoulders of a garment; and

FIG. 8 schematically illustrates the system for measuring electric signals incorporated with an audio device.

### DETAILED DESCRIPTION OF THE PREFERRED EMBODIMENTS

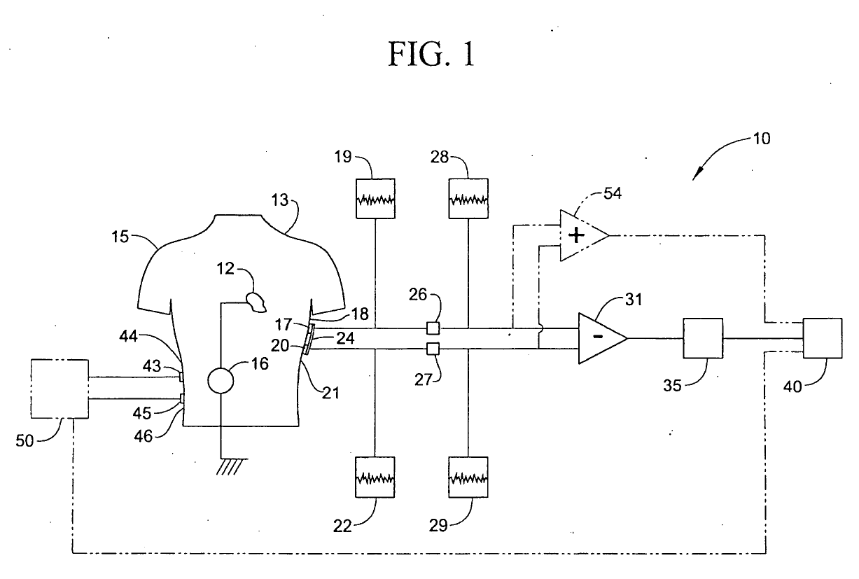

With initial reference to FIG. 1, a sensor system 10, constructed in accordance with the present invention, is arranged to measure signals from a bioelectric source 12 within a body 13 of an individual 15, such as a medical patient, animal, test subject or the like. Bioelectric source 12 creates an electric potential of interest 16 which is depicted as a cardiac signal, but could also be generated by other muscle, nerve or brain action. A first sensor 17 is placed at a first measurement location 18 so as to detect electric potential of interest 16 and generate a first electrical signal 19 possibly representative of electric potential of interest 16. Similarly a second sensor 20 placed at a second measurement location 21 near first sensor 17 so as to detect electrical potential of interest 16 and generate a second electrical signal 22 possibly representative of electric potential of interest 16. First and second sensors 17 and 20 are positioned near to each other and far enough from source 12 that signal produced by source 12 will be similar at each sensor 17, 20.

As indicated in FIG. 1, a plurality of sensors, including first and second sensors 17 and 20, could be supported by a common housing or carrier 24. This arrangement provides for convenient placement and control in the positioning of the sensors relative to each other. In addition, the use of common housing/carrier 24 assures an optimized relationship between sensors 17 and 20, while maintaining sufficient electrical and mechanical isolation to avoid coupling unwanted signals into the sensors.

The first and second signals 19, 22 from first and second sensors 17, 20 may be passed through first and second adjusters 26, 27 for altering first and second electrical signals 19, 22 respectively to compensate for changes in first and second electrical signals 19, 22 caused by placement of first and second sensors 17, 20 and electrical characteristics of first and second sensors 17, 20 to create first and second altered electrical signals 28, 29. For example, adjuster 26 preferably adjusts a gain or a time offset of electrical signal 19 to match a gain or time offset of electrical signal 22. Since amplifiers and signal adjusters are well known, the details of such electrical devices will not be discussed here. The use of first and second adjusters 26, 27 is completely optional and when adjusters 26, 27 are not used the unaltered first and second electrical signals 19, 22 are processed further as set forth below.

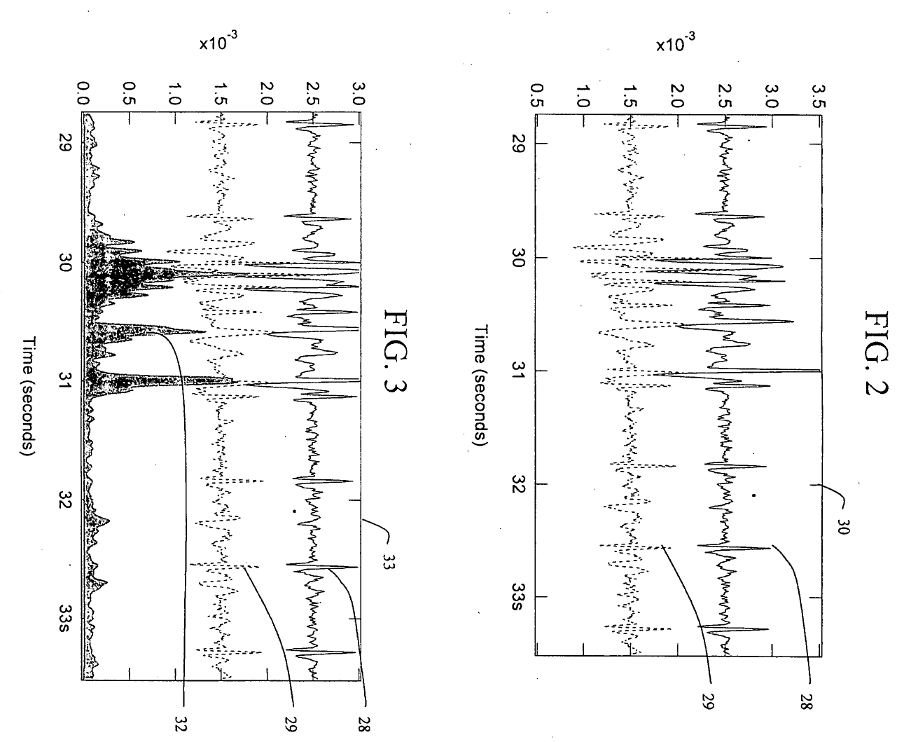

Referring now to FIG. 2, a chart 30 is shown as an example of first and second altered electrical signals 28, 29 which in this case are two ECG traces derived from first and second altered electrical signals 28, 29 obtained from first and second sensors 17, 20. The resulting altered signals 28, 29 are compared by a comparator 31 to produce a comparison result 32 which can be seen in FIG. 3 depicting a chart 33 showing the magnitude of the difference or comparison result 32 between the traces.

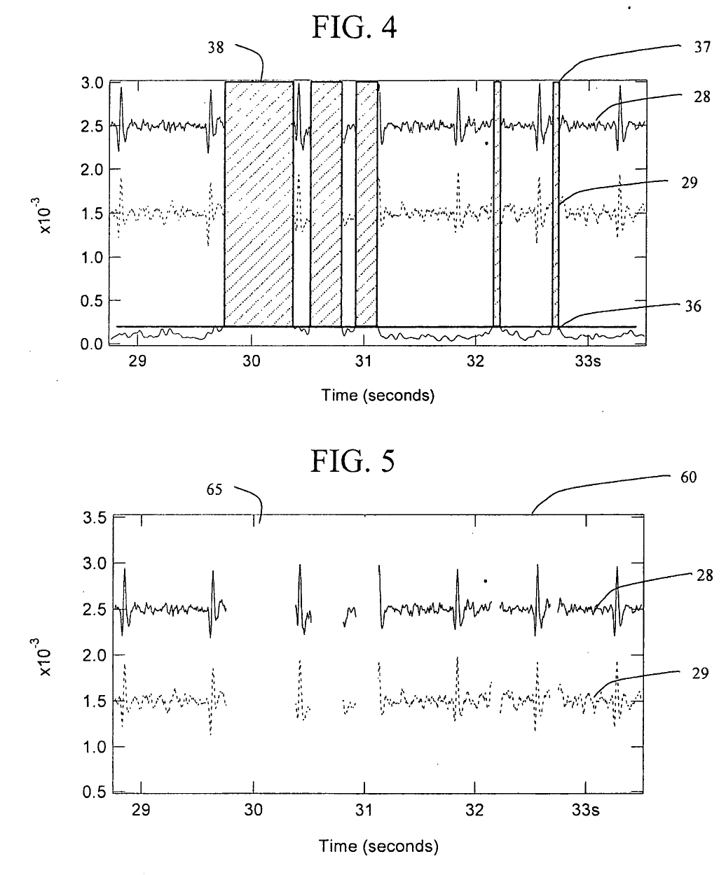

A threshold generator 35 generates a threshold 36 which can be seen in a chart 37 depicted in FIG. 4. Chart 37 indicates that threshold 36 identifies artifact regions or locally produced artifacts 38. This is done when the magnitude of comparison result 32 is compared with threshold 36 produced by threshold generator 35. Depending on implementation, threshold 36 is a static value, or is set dynamically, possibly incorporating duration information. If comparison result 32 exceeds threshold 36, an interpretation system, i.e. an electronic circuit 40 suggests that first and second electrical signals 19, 22 represent locally produced artifacts 38. In other words, if result 32 exceeds threshold 36, interpretation system 40 will indicate that the data is suspect. Otherwise, interpretation system 40 will expect that the data is reliable. While in this example the magnitude of the comparison is used in another embodiment, time offset data of the first and second electrical signals 19, 22 are used to detect locally produced artifacts.

The first and second altered signals 28, 29 can optionally be added, averaged or otherwise combined to produce a result with a better signal-to-noise ratio than either individually, with the resultant signal being used by interpretation system 40. System 10 from sensors 17, 20 onward is implemented in analog electronics, or signals 19, 22 can be digitized at any point and the further processes performed digitally.

In the embodiment above, first and second sensors 17 and 20 are at positions 18 and 21 in proximity to each other. It should be noted that positions 18 and 21 could effectively be the same position if, for instance, sensors 17 and 20 contacted body 13 at a plurality of discrete locations and those locations for sensor 17 were interleaved with those of sensor 20 such that the average of the contact locations for each of the two sensors was essentially the same location.

In the embodiment above, first and second sensors 17 and 20 are of the same kind, with differing positions, i.e., as stated above, first sensor 17 is located at first measurement location 18 and second sensor 20 is located at second measurement location 21. Since the difference in position is small compared with the location of source 12, the signal from source 12 will present similarly to each of sensors 17, 20. However, other signal sources from within sensors 17, 20 themselves, the interface between sensors 17, 20 and individual 15, within individual 15 in proximity to one or the other sensor 17, 20, or other causes will not present similarly to each sensor 17, 20, and will therefore create a signal difference which can be identified by system 10. Note that system 10 in each case is used to determine whether or not an observed signal originates from intended signal source 12, or more locally to sensors 17, 20.

Alternatively, sensors 17 and 20 are co-located or closely located, yet receive signals in differing ways. For instance, they might be capacitive sensors with differing stand-offs from the skin. As such, they would respond similarly to distant intended signal source 12, but very differently to signals or artifacts 38 generated at or near the skin. The processing chain is the same, except that the signal-scaling elements, i.e., adjusters 26 and 27, would model the differences in sensor response between sensors 17 and 20.

Additionally, system 10 can be implemented with more than two sensors, such as including an additional sensor (not shown), also in proximity to sensors 17 and 20. Signals can then be compared in pairs through the same process described above. Just as signals deriving from sensors 17 and 20 are compared to infer whether either of them contains signals that are locally generated, so can signals deriving from sensor 17 and the additional sensor be compared, and likewise signals deriving from sensor 20 and the additional sensor can be compared. If more than three sensors are used, more pairwise combinations can be established. If the number of sensors in proximity is N, the number of pairwise comparisons possible will be [N*(N−1)/2]. The signals from any pair(s) of sensors for which the comparison suggests that the signals are not locally generated can then be analyzed individually, or combined by averaging or other techniques.

If the location or characteristics of the locally generated signal are of interest, they can be determined by this invention. For instance, in the example above, if the comparisons between sensor 17 and the additional sensor, and between sensor 20 and the additional sensor both suggest the presence of a locally-generated signal, yet the comparison between sensors 17 and 20 suggests no locally-generated signal, then the locally-generated signal will be concluded to have been measured by the additional sensor.

Additionally, since useful bioelectric signals are generally formed by a difference between sensors that see differing presentations of bioelectrical source 12, additional instances of the invention can be implemented using another set of sensors not in proximity to those of system 10. For example, as shown in FIG. 1, an additional system (not separately labeled), similar to system 10, includes a third sensor 43 placed at a measurement location 44 so as to detect electric potential of interest 16 and generate a third electrical signal possibly representative of electric potential of interest 16. A fourth sensor 45 placed at a fourth measurement location 46 near third sensor 43 so as to detect electrical potential of interest 16 and generate a fourth electrical signal possibly representative of electric potential of interest 16.

A series of electronic devices 50 located downstream of third and fourth sensors 43, 45 are represented as a box. Electronic devices 50 optionally include third and fourth adjusters and also a second comparator 54 for comparing third and fourth altered electrical signals to produce a second comparison result, as well as a second electronic circuit for determining if the second comparison result suggests that the third and fourth electrical signals represent electric potential of interest 16 or locally produced artifacts. In such a system, data can be inferred to reflect intended source 12 if any pair of sensors that agree sufficiently well. More sensors improve the probability that such a pair will exist. Multiple such pairs can be considered individually, or averaged together to form a composite signal as represented by a line going from electronic devices 50 to interpretive system 40. Interpretive system 40 can compare signals which the additional system infers are not locally generated with signals which system 10 infers are not locally generated to obtain a view of intended source 12 from differing perspectives. In either case, interpretive system 40 can remove locally produced artifacts 38 from electrical signals 28, 29 as shown in FIG. 5 on chart 60 at signal portions 65.

Using electrode technologies alternative to traditional wet electrodes, a more comfortable and less intrusive arrangement is employed to collect signals 19, 20 from subject 15. When such arrangements produce high quality data, they are used for detailed medical analysis. However, if the data is of lower quality, relatively simple measurements such as heart rate are extracted. Either type of arrangement benefits greatly by sensor system 10, as described above, for determining when data is reliable verses unreliable.

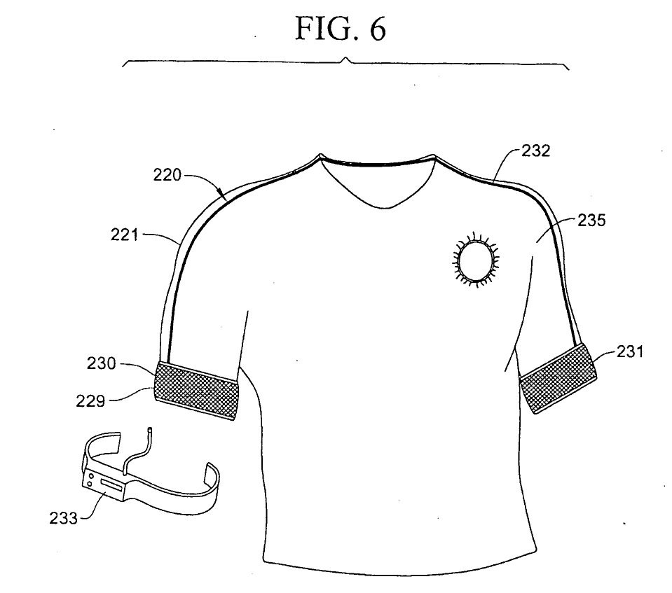

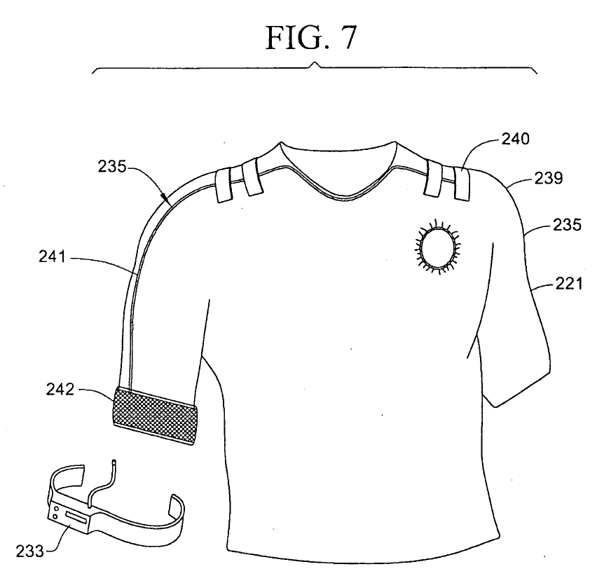

FIG. 6 depicts another preferred embodiment in which a sensor system 220 is incorporated into a shirt or garment 221. Sensors 229 that produce a vector across a heart zone are embedded in two armbands 230, 231, which might be part of shirt 221, or attached to shirt 221. Attachable armbands 230, 231 preferably have a common ground. Multiple sensors 229 can be incorporated in each armband 230, 231, with sensor system 10 being used to infer when sensors 229 are providing useable data. As in the previous description, sensor system 220 may use individual sensors or may incorporate a plurality of discrete sensors 229. Electrical connections 232 between sensor armbands 230, 231 may optionally be incorporated into shirt 221. Similarly, an electronics package 233 may be detachable from or integrated with the other components.

FIG. 7 depicts another preferred embodiment in which a multiple sensor system 235 with electrodes or sensors is incorporated into conductive fabric in a shoulder 239 of shirt 221. System 235 relies on very weak resistive and weak capacitive coupling to subject 15. Capacitive sensors 240 are incorporated into shoulders 239 or arm bands 242 of shirt 221, with the weight of shirt 221 holding sensors 240 to subject 15. Sensor system 10 is used to infer which sensors 240 are providing useable data. These sensors 240 are preferably held in contact with or near body 13 merely by the weight of shirt 221 and the multiplicity of sensors 240, in combination with sensor system 10, allows the determination of which sensors 240 are providing valid signals at any given time. Again, there are electrical connections 241 incorporated into shirt 221, and an integrated or detachable electronics package 233 is provided. Although FIG. 7 depicts sensor package 233 at sleeve 242, it can be placed wherever most convenient.

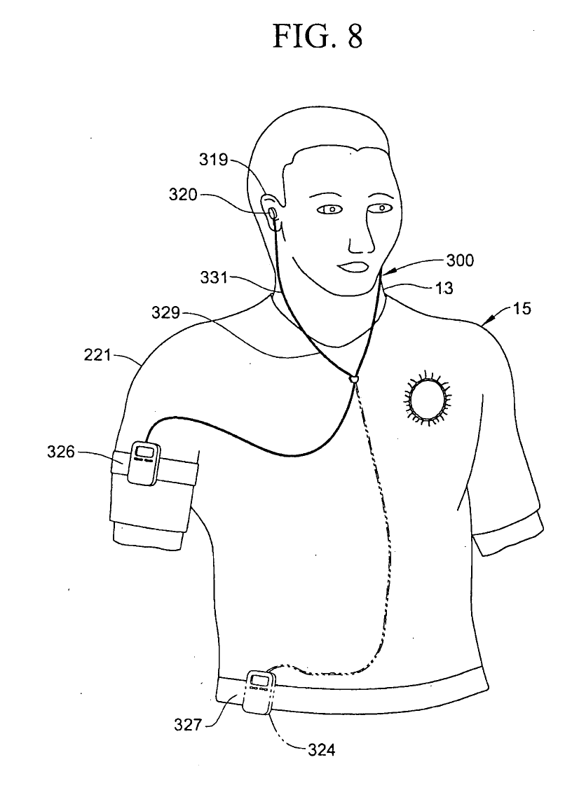

Finally, FIG. 8 depicts another preferred embodiment of the invention including a system 300 of sensors, which utilizes body attachments already commonly made with portable music players to form a MP3/CD/Radio ECG system to make a bioelectric measurement of cardiac and/or other bioelectric signals. This system 300 utilizes a hardware configuration often currently worn in an ear 319 and includes at least one earpiece 320, along with audio electronics module 324 often worn on an arm 326 or hip 327.

Wires 329 contain strands which, in addition to the usual audio functions, provide power and signal connections to electronics module 324. At this point, it should be noted that, although FIG. 8 depicts two electronics modules 324, only one would be implemented in a given system 300. For this reason, one electronics module 324 on hip 327 is shown in phantom. In any case, electronics module 324 contains a sensor system of one or more individual sensors which provides a measurement vector across the heart, enabling a cardiac signal to be measured. In addition, electronics module 324 includes sensor system 10 discussed above. Ear piece 320 preferably contains a sensor or a sensor system that measures the bioelectric potential of interest 16 at ear 319. Sensor system 300 is preferably a single sensor making a resistive, capacitive or hybrid (resistive/capacitive) connection to ear 319, or has two or more such sensors in an implementation of sensor system 10 discussed above. Because there will likely be local measurement artifacts 38 resulting from the movement of subject 15, multiple sensors may be at each location, and preferably system 10 is used to infer when the sensors are providing useable data.

Although described with reference to various preferred embodiments of the invention, it should be readily understood that various changes and/or modifications can be made to the invention without departing from the spirit thereof. In general, the invention is only intended to be limited by the scope of the following claims.

I claim:

1. A sensor system for measuring an electric potential of interest generated by a distant source in a body in the presence of locally produced artifacts comprising:
a first sensor placed at a first measurement location for detecting the electric potential of interest and generating a first electrical signal possibly representative of the electric potential of interest;
a second sensor placed at a second measurement location near the first sensor for detecting the electrical potential of interest and generating a second electrical signal possibly representative of the electric potential of interest;
a comparator for comparing the first and second electrical signals to produce a comparison result; and
an electronic circuit for determining if the comparison result suggests that the first and second electrical signals represent the electric potential of the source of interest or locally produced artifacts.

2. The sensor system according to claim 1, further comprising: first and second adjusters for altering the first and second electrical signals respectively to compensate for changes in the first and second electrical signals caused by placement of the sensors or electrical characteristics of the sensors, wherein the comparator compares the first and second electrical signals after the first and second electrical signals are altered.

3. The sensor system according to claim 1, further comprising: a threshold generator for setting a threshold level wherein the electronic circuit compares the comparison result of a difference between the first and second electrical signals with the threshold level to determine if the first and second electrical signals represent the electric potential of the source of interest.

4. The sensor system according to claim 3, wherein the threshold level is a static value.

5. The sensor system according to claim 3, wherein the threshold level is a dynamic value.

6. The sensor system according to claim 3, wherein the first and second electrical signals include timing data and the comparator measures a time offset of the time data between the first and second electrical signals.

7. The sensor system according to claim 2, wherein the first adjuster filters the first electrical signal to account for known changes caused to a measured electrical signal if a sensor is moved from the second measurement location to the first measurement location.

8. The sensor system according to claim 1, further comprising:
a third sensor placed at a third measurement location for detecting the electric potential of interest and generating a third electrical signal possibly representative of the electric potential of interest;
a fourth sensor placed at a fourth measurement location near the third sensor for detecting the electrical potential of interest and generating a fourth electrical signal possibly representative of the electric potential of interest; and
wherein the third and fourth electrical signals are compared to produce a second comparison result and the second comparison result is used to determine if the third and fourth electrical signals represent the electric potential of the source of interest or locally produced artifacts.

9. The sensor system of claim 1, further comprising:
an ear piece which contains at least one of the first and second sensors;
an audio generating device for creating audio signals worn in proximate contact to the body; and
cables carrying the audio signals and the first and second electrical signals from the sensors to the comparator and the electronic circuit.

10. The sensor system of claim 1, further comprising: a garment, wherein the sensors are incorporated into the garment.

11. The sensor system of claim 10, wherein the garment has arms and the sensors are incorporated into the arms of the garment.

12. The sensor system of claim 10, wherein the sensors are removably attached to the garment.

13. The sensor system of claim 10, wherein the electronic circuit is readily detachable from the garment.

14. The sensor system according to claim 10, wherein the garment has shoulders and the sensors are incorporated into the shoulders of the garment.

15. The sensor system according to claim 1, further comprising: a common carrier, wherein the first and second sensors are supported by the common carrier.

16. A sensor system for measuring an electric potential of interest generated by a distant source in a body in the presence of locally produced artifacts comprising:
a first sensor placed at a first measurement location for detecting the electric potential of interest and generating a first electrical signal possibly representative of the electric potential of interest; and
a second sensor placed at a second measurement location near the first sensor for detecting the electric potential of interest and generating the second electrical signal possibly representative of the electric potential of interest;
means for comparing the first and second signals and generating a comparison result; and
means for determining if the comparison result suggests that the first and second signals are sufficiently similar to each other that they describe the electrical potential of interest as opposed to some locally produced artifacts.

17. The sensor system according to claim 16, further comprising:
means for adjusting the first and second signals to compensate for differences between the sensors due to differing individual characteristics or placement wherein the comparing means compares the first and second electrical signals after the first and second electrical signals are altered.

18. The sensor system according to claim 16, further comprising:
means for establishing a threshold level; and
means for comparing the comparison result with the threshold level to determine whether or not the first and second electrical signals represent the electric potential of interest.

19. The sensor system according to claim 18, wherein the first and second electrical signals include timing data and wherein the sensor system further comprises means for determining if time offset data in the first and second electrical signals provided by the two sensors indicates that they describe the source of interest as opposed to locally produced artifacts.

20. The sensor system according to claim 16, further comprising:
means for filtering the first electrical signal to account for known changes in an electrical signal caused by moving a sensor from the second measurement location to the first measurement location.

21. The sensor system according to claim 16, further comprising: a common carrier, wherein the first and second sensors are supported by the common carrier.

22. A method of measuring an electric potential of interest generated by a relatively distant source in a body in the presence of locally produced artifacts comprising:
generating a first electrical signal at a first measurement location possibly representative of the electric potential of interest; and
generating a second electrical signal at a second measurement location possibly representative of the electric potential of interest;
comparing the first and second electrical signals to produce a comparison result; and
determining from the comparison result which portions of the first and second electrical signals actually represent the electrical potential of interest and which portions represent locally produced artifacts.

23. The method of claim 22, further comprising:
adjusting the first and second electrical signals to compensate for differences in sensor electrical characteristics and creating first and second altered electrical signals, wherein the first and second altered electrical signals are compared after the first and second electrical signals are altered.

24. The method of claim 22, further comprising: developing a threshold level and comparing the comparison result with the threshold level to determine if the signal represents the electrical potential of the source of interest or locally produced artifacts.

25. The method of claim 24, further comprising: determining a time offset of the first and second electrical signals.

26. The method of claim 22, further comprising: filtering data from the first electrical signal to account for known changes caused to a measured signal if a sensor generating the second electrical signal is moved from the second measurement location to the first measurement location.

27. The method of claim 22, further comprising:
removing portions of the first and second signals that represent locally produced artifacts;
generating a third electrical signal at a third measurement location possibly representative of the electric potential of interest; and
generating a fourth signal at a fourth measurement location possibly representative of the electric potential of interest;
comparing the third and fourth electrical signals to produce a second comparison result;
determining from the second comparison result which portions of the third and fourth electrical signals actually represent the electrical potential of interest and which portions represent locally produced artifacts;
removing portions of the third and fourth signals that represent locally produced artifacts; and
combining portions of the first and second signals that represent the electric potential of interest with portions of the third and fourth signals that represent the electric potential of interest to produce a composite signal that represents the electric potential of interest.

28. The method of claim 22, further comprising:
supporting first and second sensors, which generate the first and second electrical signals respectively, on a common housing or carrier.
